(19) United States
(12) Patent Application Publication
(10) Pub. No.: US 2009/0280193 A1
(43) Pub. Date: Nov. 12, 2009

(54) Enerceutical mediated activation of the Alternative Cellular Energy (ACE) pathway in the therapy of diseases

(75) Inventor: William John Martin, South Pasadena, CA (US)

Correspondence Address: William John Martin, 1634 Spruce Street, South Pasadena, CA, 91030, US

(73) Assignee: MI Hope Inc., South Pasadena, CA (US)

(21) Appl. No.: 12/151,779

(22) Filed: May 8, 2008

(51) Int. Cl. A61K 33/14; A61K 33/18; A61K 36/00; A61P 43/00

(52) U.S. Cl. 424/663; 424/725; 424/667

(57) ABSTRACT

Alternative cellular energy pigments (ACE-pigments) provide a source of cellular energy other than that provided through the oxidative metabolism of foods, or in the case of plants and certain bacteria, through the process of photosynthesis. In some patients, ACE pigments exist in a form that can be further energized or activated using ultraviolet (UV) light, especially if the reaction is initially triggered by the presence of suitable dyes, such as neutral red. A method is described to further enhance the activation of the ACE pathway in humans and animals deprived of ACE. The method comprises using natural or man-made sources of ACE products (enerceuticals), with or without the inclusion of a suitable dye, such as neutral red; or other activating components, such as magnesium chloride; with the combined mixture being put into a UV transparent container, such as a plastic bag, or placed on some other material, which can be laid onto the skin and illuminated with a UV light source. The process of activating the ACE pathway is evidenced by UV inducible fluorescence seen within areas of the patients' skin and/or mucus membranes. This fluorescence fades as the ACE pathway becomes fully activated. Activating the ACE pathway can have therapeutic benefits in various infectious and non-infectious diseases including illnesses attributed to infections with stealth adapted and conventional viruses and diseases attributed to an inadequacy of the mitochondria and associated metabolic pathways, including states of hypoxia and nutritional deficiencies.

### CROSS REFERENCE TO RELATED APPLICATIONS

### Co-Pending Patent Application

### Methods for Detection of Ultraviolet Light Reactive Alternative Cellular Energy Pigments (ACE-pigments) William John Martin Submitted Dec. 24, 2007

### Method of Activating the Alternative Cellular Energy (ACE) Pathway in the Therapy of Herpes Virus Infections William John Martin and Sheila Calderon Submitted Dec. 26, 2007

Method of Assessing and of Activating the Alternative Cellular Energy (ACE) Pathway in the Therapy of Diseases. William John Martin Submitted Jan. 17, 2008

### Previously Submitted but Now Abandoned Patent Applications

Ser. No. 10/044,683. Therapy of stealth virus associated cancers and other conditions using light. William John Martin. (Abandoned)
Ser. No. 10/047,313. Therapy of stealth virus associated cancers and other conditions using medium chain triglycerides. William John Martin. (Abandoned)
Ser. No. 10/050,232. Diagnosing and monitoring the therapy of stealth virus infections based on the detection of auto-fluorescent material in hair. William John Martin. (Abandoned)
Ser. No. 10/058,480. Therapy of stealth virus associated cancers and other conditions using magnetic energy. William John Martin. (Abandoned)
Ser. No. 10/164,258 Energy supportive therapy of stealth virus associated diseases. William John Martin. (Abandoned)
Ser. No. 10/174,466 Sound therapy of stealth virus associated diseases. William John Martin. (Abandoned)
Ser. No. 10/192,936 ACE-Pigments and humic acids as energy sources. William John Martin. (Abandoned)

### Submitted: 10/______ Methods for Collection of Alternative Cellular Energy Pigments (ACE-pigments). William John Martin. (Abandoned)

Submitted: 10/______ Methods for Elimination of Toxic Alternative Cellular Energy Pigments (ACE-pigments) and for Their Replacement Using Activated Humates, including Humic and Fulvic Acids. William John Martin. (Abandoned)

### United States Patents (Awarded)

U.S. Pat. No. 5,985,546 Stealth virus detection in the chronic fatigue syndrome. William John Martin
U.S. Pat. No. 5,891,468 Stealth virus detection in the chronic fatigue syndrome. William John Martin
U.S. Pat. No. 5,753,488 Isolated stealth viruses and related vaccines. William John Martin
U.S. Pat. No. 5,703,221 Stealth virus nucleic acids and related methods. William John Martin

### PCT (Patent Cooperation Treaty)

WO 92/20797 Stealth virus detection in the chronic fatigue syndrome. William John Martin
WO 99/34019 Stealth virus nucleic acids and related methods. William John Martin
WO 99/60101 Stealth viruses and related vaccines. William John Martin

### REFERENCES TO PUBLISHED ARTICLES

### Alternative Cellular Energy Pigments (ACE-Pigments):

1 Martin W J. Alternative cellular energy pigments mistaken for parasitic skin infestations. Exp. Mol. Path 78: 212-214, 2005.
2 Martin W J. Alternative cellular energy pigments from bacteria of stealth virus infected individuals. Exp. Mol. Path 78: 217-217, 2005.
3 Martin W J. Progressive Medicine. Exp Mol Path 78: 218-220, 2005.
4 Martin W J, Stoneburner J. Symptomatic relief of herpetic skin lesions utilizing an energy based approach to healing. Exp. Mol. Path 78: 131-4, 2005.
5 Martin W J. Etheric Biology. Exp Mol Path 78: 221-227, 2005.
6 Martin W J. Stealth Virus Culture Pigments: A Potential Source of Cellular Energy. Exp. Mol. Pathol. 74: 210-223, 2003.
7 Martin W J. Complex intracellular inclusions in the brain of a child with a stealth virus encephalopathy. Exp. Mol. Pathol. 74: 179-209, 2003.
8 Martin W J. Photons and phonons: Theoretical aspects of biophysics and potential therapeutic applications. Proceeding of Neural Therapy Workshop on Sound and Light Therapy, Seattle, Wash., Feb. 21-23, 2003.

### Stealth Adapted Viruses

1 Martin W J Chronic fatigue syndrome among physicians. A potential result of occupational exposure to stealth viruses. Explore 2001; 10: 7-10.
2 Martin W J. Stealth Viruses. Explore 2001; 10: 17-19.
3 Durie G M, Collins R. Martin W J. Positive stealth virus cultures in multiple myeloma. A possible explanation for neuropsychiatric co-morbidity. Presented at the Am. Soc. Hematology annual meeting October 2000.
4 Martin W J. Chemokine receptor-related genetic sequences in an African green monkey simian cytomegalovirus-derived stealth virus. Exp Mol Pathol. 2000; 69:10-6.
5 Martin W J., Anderson D. Stealth virus epidemic in the Mohave Valley: severe vacuolating encephalopathy in a child presenting with a behavioral disorder. Exp Mol Pathol. 1999; 66:19-30.
6 Martin W J. Melanoma growth stimulatory activity (MGSA/GRO-alpha) chemokine genes incorporated into an African green monkey simian cytomegalovirus-derived stealth virus. Exp Mol Pathol. 1999; 66:15-8.
7 Martin W J. Bacteria-related sequences in a simian cytomegalovirus-derived stealth virus culture. Exp Mol Pathol. 1999; 66:8-14.
8 Martin W J. Stealth adaptation of an African green monkey simian cytomegalovirus. Exp Mol Pathol. 1999; 66:3-7.
9 Martin W J. Cellular sequences in stealth viruses. Pathobiology 1998; 66:53-8.
10 Martin W J. Detection of RNA sequences in cultures of a stealth virus isolated from the cerebrospinal fluid of a health care worker with chronic fatigue syndrome. Case report. Pathobiology. 1997; 65:57-60.
11 Martin W J., Anderson D. Stealth virus epidemic in the Mohave Valley. I. Initial report of virus isolation. Pathobiology. 1997; 65:51-6.
12 Martin W J. Simian cytomegalovirus-related stealth virus isolated from the cerebrospinal fluid of a patient with bipolar psychosis and acute encephalopathy. Pathobiology. 1996; 64:64-6.
13 Martin W J. Stealth viral encephalopathy: report of a fatal case complicated by cerebral vasculitis. Pathobiology. 1996; 64:59-63.
14 Martin W J. Genetic instability and fragmentation of a stealth viral genome. Pathobiology. 1996; 64:9-17.
15 Martin W J. Severe stealth virus encephalopathy following chronic-fatigue-syndrome-like illness: clinical and histopathological features. Pathobiology. 1996; 64:1-8.
16 Martin W J. Stealth virus isolated from an autistic child. J Autism Dev Disord. 1995; 25:223-4.
17 Gollard R P, Mayr A., Rice D A, Martin W J. Herpesvirus-related sequences in salivary gland tumors. J Exp Clin Cancer Res., 1996; 15: 1-4.
18 Martin W J., Glass R T. Acute encephalopathy induced in cats with a stealth virus isolated from a patient with chronic fatigue syndrome. Pathobiology. 1995; 63:115-8.
19 Martin W J, et al. African green monkey origin of the atypical cytopathic ‘stealth virus’ isolated from a patient with chronic fatigue syndrome. Clin Diag Virol 1995: 4: 93-103.
20 Martin W J. Stealth viruses as neuropathogens. CAP Today. 1994; 8: 67-70.
21 Martin W J. et al. Cytomegalovirus-related sequence in an atypical cytopathic virus repeatedly isolated from a patient with chronic fatigue syndrome. Am J. Pathol. 1994; 145: 440-51.
22 Martin W J. Activation of the alternative cellular energy (ACE) pathway as natural therapy for patients with autism. Submitted Mar. 27, 2008 to J. Autism and Developmental Disorders.
23. Martin W J. Activation of the alternative cellular energy (ACE) pathway as natural therapy for herpes simplex and herpes zoster virus infections. Submitted Mar. 27, 2008 to J. Infectious Diseases and subsequently to J. Complimentary and Alternative Medicine.

### STATEMENT REGARDING FEDERALLY SPONSORED RESEARCH OR DEVELOPMENT

Not applicable: No Federal finding was received in support of this patent application.

### REFERENCE TO SEQUENCE LISTING, A TABLE OR A COMPUTER PROGRAM LISTING COMPACT DISK APPENDIX

Not applicable.

### BACKGROUND OF THE INVENTION

The invention is based on the following broad conceptual understanding on how the body can acquire cellular energy other than through the oxidative metabolism of foods. Essentially, an alternative cellular energy (ACE) pathway has been identified that is mediated by the energy converting (transducing) properties of mineral containing complexes of organic molecules, arbitrarily termed alternative cellular energy pigments (ACE pigments) when derived from patients. Cellular energy generated by this pathway can seemingly complement the chemical energy that is derived by living organisms from the metabolism of food. The ACE pathway is likely to contribute to various physiological functions of the body. Of particular relevance to this application, the ACE pathway is postulated to provide an auxiliary defense mechanism beyond that of the immunological system, such that an inadequacy of this pathway may limit the body's capacity to overcome various infectious diseases. The ACE pathway is also anticipated to be involved in the normal functioning of many organs, including the brain. Moreover, it is reasonable to presume that many illnesses, not necessarily of infectious origin, may place an added burden on the ACE pathway and that an inadequacy, deprivation or excessive demands on the ACE pathway may be a factor in delaying the normal disease recovery process. Conversely, augmenting or activation of an impaired ACE pathway may facilitate recovery from a wide range of various illnesses. Moreover, a fully functioning ACE pathway is likely to also be a factor in disease prevention, maintaining optimal wellness, enhancing athletic performance, increasing cognitive abilities, etc.

ACE pigments are envisioned as tiny batteries that can be either fully charged or not fully charged with energy. In the latter state, ACE pigments can accept additional energy from various sources, including ultraviolet (UV) light. Absorption of UV light energy can be observed as the emission of visible light; a process known as fluorescence. In some patients, including patients in whom particles of ACE pigments are misdiagnosed as parasites (delusional parasitosis or Morgellon's disease) direct fluorescence of ACE pigments can be readily observed. In many patients, however, the absorption of UV light energy and the resulting fluorescence by inadequately charged ACE pigments can be strongly enhanced in the presence of certain dyes, including neutral red. ACE pigments can also interact with various other dyes, including the fluorescent dye acridine orange, yielding a multiplicity of colors that are well beyond the narrow spectrum of green light emitted solely by UV illuminated acridine orange. Once a fluorescence reaction is evoked, nearby ACE pigments and other chemicals, without any direct contact with the dye, will also commonly undergo reactions that presumably reflect a physical energy transfer mechanism.

The potential linking and/or association between chemical reactions and either the input or the emission of physical energies has not been as extensively studied as have the independent disciplines of biochemistry and biophysics. Photosynthesis provides a model system for the conversion of the energy contained within visible sunlight into chemical bonding energy stored within carbohydrates. Other forms of electromagnetic radiation can be shown to facilitate various other types of chemical reactions. It follows, therefore, that electromagnetic or other forms of energy emissions from a chemical reaction, or from energy transduction (conversion) by chemicals, can potentially have chemical or energizing effects on reactants that are physically separated from the energy source. Along with my studies, I have not uncommonly observed unusual behavior of unicellular microbes when in the vicinity, but without actual physical contact, with a chemical reaction. I have also observed that fine particles in a neutral red solution will unexpectedly undergo marked movements when placed near UV illuminated mineral rich Lidocaine (xylocaine) containing herbal solutions that are rendered fluorescent with neutral red and other dyes. More impressively, the distinctive multi-point crystallization pattern of the neutral red that can be seen occurring in some of these solutions will also occur in a dilute solution of neutral red that is simply held in close proximity to a fluorescing solution. Such a reaction is not seen when neutral red solutions are simply exposed to UV light.

This indirect energy emitting process, triggered by a neutral red dye triggered reaction with body-derived materials termed ACE pigments, has been successfully used in expediting the healing of active skin lesions caused by herpes simplex virus (HSV) and herpes zoster virus (HZV) infections. One can either use ACE pigments collected from the active lesions of the patients or a suitable formulated equivalent of ACE pigments as the source of emitted energy. ACE pigments can commonly be retrieved from the skin of patients with a history of recurrent herpes virus lesions, as well as other diseases, and can be induced to fluoresce using neutral red. Patient derived ACE pigments or preferably a suitable formulated equivalent of ACE pigments have now been successfully used in preventing future outbreaks in patients with a prior history of recurrent herpes who were treated during a period when no active lesion was present.

As noted above patients with various other illnesses will commonly display ACE pigments in skin and body secretions that will fluoresce under UV light illumination in the presence of freshly prepared solution of neutral red dye. Some of these additional illnesses have been attributed to infections by stealth adapted viruses that fail to provoke an anti-viral cellular immune response. Major categories of these illnesses include the chronic fatigue syndrome, fibromyalgia, psychiatric and both acute and chronic neurological illnesses, autism and both behavioral and learning disorders in children, some cases of cancer and many cases of general debilitating illness. Another grouping of illnesses includes patients in whom the mitochondria and associated metabolic energy pathways are deficient, because of hypoxia (as in emphysema), nutritional deficiencies, metabolic poisoning or genetic abnormalities. An assessment of the ACE pathway can be made by determining whether material that fluoresces upon the addition of neutral red can be obtained from skin, saliva, urine, or other bodily fluids or tissues of the patients. Such observations have led to the development of simple methods to assess a patient's ACE pathway and to provide a clinical indication for efforts to recharge this pathway. These methods include the application of neutral red, either directly to the patient's skin or onto an absorbent cloth on which ACE pigments were collected from the patient by simply rubbing the cloth onto skin areas. Using this approach it was noted that many substances exist in Nature that will fluoresce if mixed with neutral red and illuminated with a UV light. It appears that the ACE pathway is a fundamental process in Nature and conceivably a forerunner to more selective processes such as photosynthesis. Organically complexed minerals, such as magnesium within chlorophyll, are presumed to be critical elements in the capture and processing of external physical energies.

This concept is fully consistent with the finding that various mineral and organic substances can be formulated that similarly show fluorescence when mixed with neutral red and illuminated with UV light. Not all cellular energy delivering products show this reaction but may yet presumably still be are able to absorb energies; and further that such absorption may be facilitated using various types of dyes or other chemicals. The advantage of a current formulation that has been devised over the last several years is that it clearly fluoresces with neutral red and that a distant action can be easily observed in a distantly held neutral red indication solution. This action includes otherwise unexplained movements of fine particles within the neutral red solution and more strikingly the induced crystallization that occurs in the neutral red solution. The present formulation has replaced the need for collecting ACE pigments from the patient. It is also suitable as a liquid medium that when triggered to fluoresce with neutral red can be contained within a UV transparent plastic bag that can be placed onto the skin of the patient and illuminated using a UV light source. Again, this method allows for the avoidance of direct contact of the therapeutic solution with the patient's skin. The UV inducible fluorescence, or other form of energy emission, within the plastic bags will typically evoke some discernable fluorescence within at least some skin areas in most of the treated and symptomatic patients, consistent with an overall systemic activation of the body's ACE pathway. Such activation is taken as an indication that the ACE pathway in the patient is not fully charged and that the patient is likely to benefit from further activation of the pathway. The liquid-in-a-bag format also allows for the addition of other materials that can further enhance the activation and/or fluorescence of the formulations being studied. Materials under evaluation include iodine, magnesium chloride, hydrogen-rich water and others chemicals. Additional background information is provided in the cited references of the inventor and these publications are included by reference herein.

### BRIEF SUMMARY OF THE INVENTION

The invention describes a method to treat a wide variety of illnesses as well as potentially enhance the overall vitality and wellbeing of individuals. Specifically, the specification describes a method of energy-based activation of an alternative cellular energy (ACE) pathway in humans as well as potentially in animals. This pathway is mediated by energy transducing materials termed alternative cellular energy pigments (ACE pigments). In some individuals, both as a cause and a consequence of disease, the ACE pigments are not sufficiently activated (or charged). This lack of full activation or charging can be inferred if patient derived ACE pigments can be shown to fluoresce under UV light illumination when contacted with freshly prepared neutral red dye. The lack of detectable fluorescing material from the skin, buccal swabs (saliva) or urine is, conversely, presumptive evidence that at least one aspect of the ACE pathway is either fully charged or not being called upon to the extent it is in patients in whom fluorescence material can be collected. Patients have been identified in whom skin-derived, neutral red inducible-UV fluorescing material was present even though at the time there was no actual skin disease observed. Neutral red inducible and occasionally unaided UV fluorescence has also been shown in hair samples and in buccal swabs obtained from patients with a variety of systemic illnesses. Skin derived ACE pigments have been collected onto Q-tips and onto absorbent gauze swabs and surgical dressings. The materials used to collect the ACE pigments were stained with neutral red and placed over the skin areas from which the material was collected, avoiding direct contact of the dye with the skin with for example an intervening Saran Wrap or plastic coated toweling. The Q-tip or other material used to collect the ACE pigments fluoresced when illuminated with UV light. Furthermore, the fluorescence on the Q-tip or other material appeared to activate a fluoresce reaction that involved the patient's skin. The fluorescence in the patient's skin gradually subsided over a 10-60 minute period. Of the various patients so treated, they have consistently reported improvements in their sense of wellbeing. This approach provided a useful method to seemingly “jump starting” the ACE pathway.

Various natural products possess ACE pigment like activities and have been termed “enerceuticals” by the inventor. They are defined as materials that are capable of transducing (converting) physical energies into an energy form that can be utilized by living cells as an alternative to chemical energy derived from the metabolism of nutrients and which have the following distinguishing characteristics: i) Can provide therapeutic benefits as well as a general vitality enhancing effects to plants, animals and humans; ii) their potential therapeutic effects are not limited or restricted to any particular type or grouping of illnesses; and iii) that do not need to specifically localize to the disease tissues since they can generate field effects that extend beyond their physical location. These criteria distinguish enerceuticals from both pharmaceuticals and nutraceuticals.

An example of an enerceutical is a terpine/terpenoid rich mixture of steam-extracted sap from Japanese cedar, cypress and pine trees and plantain plants. It is marketed as HB-101 for agricultural use and as EH-101 when used as a dietary supplement in humans. This product was simply regarded as a nutrient until the inventor drew attention to its remarkable property of still being active at 1:10,000 dilutions, delaying the spoiling of caught whole fish even when simply included in the ice used to pack the intact fish, and interacting in vigorous energy exchange reactions with tincture of iodine, leading to products that can have distant effects on microbes. Another enerceutical product was formulated from xylocaine (Lidocaine) by the addition of sodium chloride depleted mineral salts obtained from the Great Salt Lake in Utah and marketed by Marine Minerals, Inc. Electrostatically active, needle-shaped crystals form that can both directly and after interaction with tincture of iodine, affect the behavior of microbes placed in a separate water droplet on the same microscope slide as the crystals. Various additional minerals and herbal products, suggested by a review of stated components of other energy delivering commercial and investigative products, have been included in variously modified formulation of Lidocaine-containing enerceuticals. One such product has been provisionally designated Epione. The herbal components were suggested from data pertaining to two related products, one known as Carnova and the other as HANSI (now also known as Enercel). Neither of these products will fluoresce with neutral red. The Epione formulation gives a bright UV light induced orange fluorescence when mixed with freshly prepared neutral red. Lidocaine by itself will not fluoresce when mixed with neutral red. Nor will this enerceutical when it is tested without contact with neutral red. UV illumination of the neutral red stained enerceutical preparation will induce small multi-pointed crystallization and heightened movement within a dilute neutral red solution physically separated from the fluorescing solution. This method has provided a simple assay to confirm the apparent physical energy transfer from the activated enerceutical preparation.

The present invention relates to combining this particular or equivalent enerceutical with neutral red to create a UV fluorescing solution that can be placed into a plastic bag or soaked into an absorbent cloth that is simply laid onto the skin and illuminated with a UV light source, so as to activate the ACE pathway in an ACE pathway deprived human or animal. The activation process can be achieved over a time period ranging from several minutes to over an hour. The size of the plastic bagging can also vary widely; the largest being tried is 17″×11″. A smaller size is more appropriate for treating a localized lesion. The UV light source is typically one or more light bulbs or various strengths and sizes and includes the 13 watt spiral UV light that is most convenient for localized lesions.

### BRIEF DESCRIPTION OF THE DRAWINGS

Not Applicable and none included

### DETAILED DESCRIPTION OF THE INVENTION

Rather than relying upon neutral red to interact directly with ACE pigments derived from a patient, I have discovered that an enerceutical product can be used with neutral red to help initiate the activation of the body's own ACE pigments. It is also likely that the enerceutical product can function directly by itself or with various other dyes. In a preferred embodiment, however, neutral red is used to activate a discernable fluorescence within the enerceutical formulation. In a typical example, up to hundred milliliters of the selected enerceutical solution is vigorously shaken and poured into a plastic bag to provide fluid coverage over a skin surface of a patient. The bag is placed on the patient's skin. The enerceutical solution is held under UV illumination just prior to the addition of a neutral red solution in a sufficient amount to achieve marked fluorescence. The actual concentration and amount of neutral red solution used can be variable but typically is about 1 mg/ml and at about a tenth of the volume of the enerceutical product. The intent is to only use sufficient neutral red to achieve maximum UV-A induced fluorescence. After gentle mixing, the UV illumination is continued for approximately an hour with a UV light comprising either fluorescent tubes or spiral light bulbs that selectively emit UV-A light. If the patient has an existing skin lesion or a prior history of recurrent skin eruptions, the bag will be placed over the involved areas of skin. Preference is also given to areas where perspiration tends to accumulate (groin areas, axilla and scalp) nerve innervations tend to be concentrated (face, hands and feet) or over the spine. Several illuminated bags can be used in the one patient if desired and the patient can be placed inside a chamber fitted with UV-A lights. UV illumination generally continues for approximately an hour. Incidentally, the palate will also commonly show UV inducible fluorescence during the procedure with subsequent fading. A small separate container of neutral red by itself can also be placed near the fluorescing bag and subsequently examined for crystallization of the neutral red solution as a marker of energy transfer from the fluorescing solution.

A suitable enerceutical is the xylocaine-mineral-herbal formulation, tentatively called Epione. It comprises 0.1% xylocaine together with calcium chloride, potassium chloride, magnesium chloride and magnesium sulfate as minerals in an aqueous solution containing 5% ethyl alcohol. Homeopathic quantities of a solution containing the herbal products Aconite napellus, Bryonia alba, Chelidonium, Cinamium, Pulsatilla, Lachesis and Thuja, are also included. Of the various components, the calcium, potassium, magnesium and xylocaine are considered as forming the major energy transducing complexes. Fluorescence reactions within the particular enerceutical formulation can also be achieved using the already fluorescent dye acridine orange.

Additional approaches under consideration include the further addition of saturated magnesium chloride in the range of 10% of the amount of enerceutical used. The magnesium chloride forms numerous fine particles that undergo progressive waves or burst of movements somewhat similar to the vigorous reactions seen with tincture of iodine added to the enerceutical. The reactions become less intense over a period of several minutes. Magnesium flakes have also been added as a means of generating hydrogen within the enerceutical-magnesium chloride mixture. Furthermore, rather than chemical grade magnesium chloride, I have also used the marine mineral sodium chloride depleted water from the Great Salt Lake. The major residual component is magnesium chloride.

Various volunteers have been treated using the fluid-in-a-bag protocol. Subjective determination is that the method provides more energy than does the use of fluorescing towels. Improvements have been noted in the time one can hold one's breadth validating earlier findings of benefits in patients with emphysema. The bags progressively lose activity after the 1-2 hour time period presumably as a result of the observed polymerization of neutral red into fine multi-spiked crystals. It is anticipated that further refinements of the protocol will be made as clinical studies continue. One such refinement is to substitute sunlight as the source of UV illumination.

The described approach is suitable for self-administration and certainly for a parent to administer to a child, including a child with autism, ADHD, etc. In conformity with FDA regulations various participants can be qualified as their own clinical investigator or this relatively simple procedure.

The principles, preferred embodiments and modes of operation of the present invention have been described in the foregoing specification. The invention which is intended to be protected herein, however, is not to be construed as limited to the particular enerceutical or dye disclosed, since they are to be regarded as illustrative rather than restrictive. Nor is the invention intended to be necessarily restricted to UV light as the source of physical energy. In some protocols, exposure to sunlight may suffice. Moreover, it is likely that portions of the electromagnetic spectrum other than UV light will prove to be useful with enerceuticals, with or without the inclusion of a dye, for activation of the ACE pathway in an ACE energy-deprived subject. Nor is the invention dependent upon the precise decision as to what does or does not constitute an enerceutical. Basically, the claims relate to the broader issue of achieving therapeutic benefit through activation of the ACE pathway with the description of the currently preferred and practiced method. Additional advantages and modifications will readily occur to those skilled in the art and especially upon practicing the currently described methods. Variations and changes may be made without departing from the spirit of the invention encompassed by the appended claims.

What I claim as my invention is:

1. A method for activating the alternative cellular energy (ACE) pathway in an ACE energy deprived human or animal subject comprising the ultraviolet (UV) light illumination of an enerceutical product, used either alone or mixed with a suitable dye, with the enerceutical product or mixture being first placed in a plastic or other UV light transparent container that is laid upon the skin surface, or is held in close proximity to the skin, and illuminated with UV light for the purpose of expediting the healing of a disease process affecting the subject.

2. The method of claim 1 in which the enerceutical is a solution containing xylocaine, minerals and herbal extracts formulated in such a manner that it fluoresces under UV illumination when mixed with the dye at a concentration sufficient to achieve UV inducible fluorescence.

3. The method of claim 1 in which the activity of the enerceutical solution is further enhanced by the addition of materials, that evoke a reaction within the enerceutical solution manifested by the formation of rapidly moving microscopically discernable particles that engage in a time limited flurry of vigorous interactions.

4. The method of claim 2 in which the enerceutical product is rendered fluorescent by using a solution the dye neutral red at a concentration ranging from 0.05 to 5.0 mg/ml.

5. The method of claim 2 in which the enerceutical product is rendered fluorescent by using a solution of acridine orange at a concentration ranging from 0.05 to 5.0 mg/ml.

6. The method of claim 3 in which the added component is a solution containing magnesium chloride.

7. The method of claim 3 in which the added component is tincture of iodine.

8. The method of claim 1 in which the disease is caused by herpes simplex virus (HSV), herpes zoster virus (HZV), Epstein-Barr virus (EBV), cytomegalovirus (CMV) and human herpesviruses 6-8 (HHV6, HHV7 and HHV8).

9. The method of claim 1 in which the disease is caused by human papillomaviruses

10. The method of claim 1 in which the disease is caused by human immunodeficiency virus (HIV).

11. The method of claim 1 in which the disease is caused by human hepatitis viruses.

12. The method of claim 1 in which the diseases is presumptively caused by atypical (stealth adapted) viruses that lack antigenic components capable of activating an effective anti-viral cellular immune response

13. The method of claim 1 in which the diseases is characterized by the formation on the skin and hair of alternative cellular energy (ACE) pigments having the form of particles and fibers and that has been referred to as Morgellon's disease

14. The method of claim 1 in which the disease is autism or a related illness, including childhood learning and behavioral disorders.

15. The method of claim 1 in which the disease is a psychiatric illness.

16. The method of claim 1 in which the disease is the chronic fatigue syndrome or a related illness, including fibromyalgia and depression.

17. The method of claim 1 in which the disease is caused by hypoxia as occurs in illnesses such as emphysema, or a metabolic or nutritional deficiency.
